SEQUENCE LISTING (34 sequences)

SEQ ID NO: 1 - DNA, 201 bp, Cicer arietinum
  source 1..201: mol_type = genomic DNA
atacctttgggttttaaccaataacttttattcattaattttgcagtggcctctttcagtcattgaatcacacagcgacaaggtataaatctttcttcccatgttttataaggcgaagtaaacagcgttgcttgctaaacagcggtgtagtgcaatttgaacaaaccattattattttgcaatacataattttgtataaag

SEQ ID NO: 2 - DNA, 201 bp, Cicer arietinum
  source 1..201: mol_type = genomic DNA
atacctttgggttttaaccaataacttttattcattaattttgcagtggcctctttcagtcattgaatcacacagcgacaaggtataaatctttcttcccgtgttttataaggcgaagtaaacagcgttgcttgctaaacagcggtgtagtgcaatttgaacaaaccattattattttgcaatacataattttgtataaag

SEQ ID NO: 3 - DNA, 201 bp, Cicer arietinum
  source 1..201: mol_type = genomic DNA
cacaggcacttggttttcttagtgacttcaaattgggacactacatgaaaattcctccaaaagctatgttcctagtacaggtaagaatgtaataatggatccatagtttgccaaatatctaactcgaaagcaatacaacatatctattgggtatggctcatggtttattgataaatgtaattcaaagccacccgtggtact

SEQ ID NO: 4 - DNA, 201 bp, Cicer arietinum
  source 1..201: mol_type = genomic DNA
cacaggcacttggttttcttagtgacttcaaattgggacactacatgaaaattcctccaaaagctatgttcctagtacaggtaagaatgtaataatggatgcatagtttgccaaatatctaactcgaaagcaatacaacatatctattgggtatggctcatggtttattgataaatgtaattcaaagccacccgtggtact

SEQ ID NO: 5 - DNA, 201 bp, Cicer arietinum
  source 1..201: mol_type = genomic DNA
tttagttttcttatgttgtggcaagtctcaaatgatttgtagatcacttttgtgtttttgtggcaatgtccaatctatatgccacaataaggtgtatttgatgaaagcagaactctcgtaccttttataagatttcttttgcatatcagtgaacttgggataagatcgggcaattggcatctatttggtgcaaatgtcata

SEQ ID NO: 6 - DNA, 201 bp, Cicer arietinum
  source 1..201: mol_type = genomic DNA
tttagttttcttatgttgtggcaagtctcaaatgatttgtagatcacttttgtgtttttgtggcaatgtccaatctatatgccacaataaggtgtatttggtgaaagcagaactctcgtaccttttataagatttcttttgcatatcagtgaacttgggataagatcgggcaattggcatctatttggtgcaaatgtcata

SEQ ID NO: 7 - DNA, 201 bp, Cicer arietinum
  source 1..201: mol_type = genomic DNA
atcaacatcaccatctccaacctctttgggatcaactagtaccgaaaccaccattgaagatttattaccagggaggtggcctttcggcaaatttctagcatgctcttcagttatgttcagagtggatccaggaagtggatctgggagcgtgtggaggattcttctgttcctgctcttgttgacagtggtcgcattacgacg

SEQ ID NO: 8 - DNA, 201 bp, Cicer arietinum
  source 1..201: mol_type = genomic DNA
atcaacatcaccatctccaacctctttgggatcaactagtaccgaaaccaccattgaagatttattaccagggaggtggcctttcggcaaatttctagcacgctcttcagttatgttcagagtggatccaggaagtggatctgggagcgtgtggaggattcttctgttcctgctcttgttgacagtggtcgcattacgacg

SEQ ID NO: 9 - DNA, 201 bp, Cicer arietinum
  source 1..201: mol_type = genomic DNA
acttgaatttctcttggtaatgctcacttgtcataataaccattgataatggaatgcacattttcatcgttgttgcttttgtaacgagttaatgacataacttagaaggatttttcccagggatgactgaatcgaaagtttaatggtcagtattatggttttggatggtttaatagttcttctggaatttcaattagcttg

SEQ ID NO: 10 - DNA, 201 bp, Cicer arietinum
  source 1..201: mol_type = genomic DNA
acttgaatttctcttggtaatgctcacttgtcataataaccattgataatggaatgcacattttcatcgttgttgcttttgtaacgagttaatgacataagttagaaggatttttcccagggatgactgaatcgaaagtttaatggtcagtattatggttttggatggtttaatagttcttctggaatttcaattagcttg

SEQ ID NO: 11 - DNA, 201 bp, Cicer arietinum
  source 1..201: mol_type = genomic DNA
tgcaggtaataacagtcttgtgcatttttcattgatcctgttattggtcgtgtgtctaacattagatgtttttatttcttccagagatacggtcctccactgtcataccctcatctgaaaatccctggactgaatgctcccattcccactggagctagctttggttatcatcctggcggatggggaaaacctccagttgac

SEQ ID NO: 12 - DNA, 201 bp, Cicer arietinum
  source 1..201: mol_type = genomic DNA
tgcaggtaataacagtcttgtgcatttttcattgatcctgttattggtcgtgtgtctaacattagatgtttttatttcttccagagatacggtcctccaccgtcataccctcatctgaaaatccctggactgaatgctcccattcccactggagctagctttggttatcatcctggcggatggggaaaacctccagttgac

SEQ ID NO: 13 - DNA, 201 bp, Cicer arietinum
  source 1..201: mol_type = genomic DNA
aacaatcaaagttctaggacacaataaaaaacataaaagaccaaagattaggaaggaatagttcatatcaaaacgatttggactgcaacatcaacttattttaagtctcttcaaccgcgattcaggccacatcattcacacatattactacgattctatgcaatattaaagttcgcaacacagccacaatttaaagctatg

SEQ ID NO: 14 - DNA, 201 bp, Cicer arietinum
  source 1..201: mol_type = genomic DNA
aacaatcaaagttctaggacacaataaaaaacataaaagaccaaagattaggaaggaatagttcatatcaaaacgatttggactgcaacatcaacttattctaagtctcttcaaccgcgattcaggccacatcattcacacatattactacgattctatgcaatattaaagttcgcaacacagccacaatttaaagctatg

SEQ ID NO: 15 - DNA, 201 bp, Cicer arietinum
  source 1..201: mol_type = genomic DNA
ccaaaagaattgattttttattttggcggtggtgtttttgttatgcagtttttaagtacaacaaaggatcggattcagtgttgttggtaaacaaacaagagtatgattcatgcaacacaaagaatccaattaccaagatggacggtggagattcaattttcactttggataaatcaggtccatttttcttcattagtggca

SEQ ID NO: 16 - DNA, 201 bp, Cicer arietinum
  source 1..201: mol_type = genomic DNA
ccaaaagaattgattttttattttggcggtggtgtttttgttatgcagtttttaagtacaacaaaggatcggattcagtgttgttggtaaacaaacaagactatgattcatgcaacacaaagaatccaattaccaagatggacggtggagattcaattttcactttggataaatcaggtccatttttcttcattagtggca

SEQ ID NO: 17 - DNA, 201 bp, Cicer arietinum
  source 1..201: mol_type = genomic DNA
gtaagaaaagtgggagatagttaattaagtaaagtgtgacaccaagttgcgccatatttgtgaaggaatggagccagagatttataaagtggttgggatacagaaagtagaagaaaggtgttggacttggaacaagatttattttgttataaaaaacaccaacaagatttaaaacttatctaacccttaggttgaattgtg

SEQ ID NO: 18 - DNA, 201 bp, Cicer arietinum
  source 1..201: mol_type = genomic DNA
gtaagaaaagtgggagatagttaattaagtaaagtgtgacaccaagttgcgccatatttgtgaaggaatggagccagagatttataaagtggttgggatagagaaagtagaagaaaggtgttggacttggaacaagatttattttgttataaaaaacaccaacaagatttaaaacttatctaacccttaggttgaattgtg

SEQ ID NO: 19 - DNA, 201 bp, Cicer arietinum
  source 1..201: mol_type = genomic DNA
tcaatgcatataaattaaattctaacgagaatacttatcagttatcactattagtgctttaatctcactattgaacttagttatttgatctccaaagggcctgtgtaacttacactttctgaaaattgtggactaggaattcctgcagctaaggatctaaacagaggggtaacttgaagaggggaactgaactctgtactt

SEQ ID NO: 20 - DNA, 201 bp, Cicer arietinum
  source 1..201: mol_type = genomic DNA
tcaatgcatataaattaaattctaacgagaatacttatcagttatcactattagtgctttaatctcactattgaacttagttatttgatctccaaagggcttgtgtaacttacactttctgaaaattgtggactaggaattcctgcagctaaggatctaaacagaggggtaacttgaagaggggaactgaactctgtactt

SEQ ID NO: 21 - DNA, 201 bp, Cicer arietinum
  source 1..201: mol_type = genomic DNA
tgtcaaatcttgatcataaattttcctttttagttgtgacacataccacttagagttacttctaagaacgcatatcatctgatacttcacttgtatcagctgacgacatctatggatgtagacgttgacgtaaagacacaaacatgagttttttttgtcaaaccaagtatcttctgatcttcagtaaccgaatgatgttaa

SEQ ID NO: 22 - DNA, 201 bp, Cicer arietinum
  source 1..201: mol_type = genomic DNA
tgtcaaatcttgatcataaattttcctttttagttgtgacacataccacttagagttacttctaagaacgcatatcatctgatacttcacttgtatcagccgacgacatctatggatgtagacgttgacgtaaagacacaaacatgagttttttttgtcaaaccaagtatcttctgatcttcagtaaccgaatgatgttaa

SEQ ID NO: 23 - DNA, 201 bp, Cicer arietinum
  source 1..201: mol_type = genomic DNA
agattataatttttaatattgaaaaaaatattaaatatattgaatgtatgcatgtatataaaattaaaaaatgacaagaatattagtagcggaacaagtttggacgcaggtgaatatcaatacaccaaaatccgtctctactcctaagttttaatttcgaaaaaaattcatacgcaatcaaaataaatttctccatctaaa

SEQ ID NO: 24 - DNA, 201 bp, Cicer arietinum
  source 1..201: mol_type = genomic DNA
agattataatttttaatattgaaaaaaatattaaatatattgaatgtatgcatgtatataaaattaaaaaatgacaagaatattagtagcggaacaagttcggacgcaggtgaatatcaatacaccaaaatccgtctctactcctaagttttaatttcgaaaaaaattcatacgcaatcaaaataaatttctccatctaaa

SEQ ID NO: 25 - DNA, 163 bp, Cicer arietinum
  source 1..163: mol_type = genomic DNA
cacctgttattgatgttactcaatttggtttcttcaagcttttgggtaaaggtgttttgcctcagaatcagccatttgttgtgaagactaaactcatttcgaagatcgctgagaagaagattaaggaagccggtggtgctgttgttcttaccgcttaattttt

SEQ ID NO: 26 - DNA, 163 bp, Cicer arietinum
  source 1..163: mol_type = genomic DNA
cacctgttattgatgttactcaatttggtttcttcaagcttttgggtaaaggtgttttgcctcagaatcagccatttgttgtgaagactaaactcatttccaagatcgctgagaagaagattaaggaagccggtggtgctgttgttcttaccgcttaattttt

SEQ ID NO: 27 - DNA, 201 bp, Cicer arietinum
  source 1..201: mol_type = genomic DNA
gaagacaatgatggctatgcttctgaaactgacgatgacaaccacaaatatgacgacgaatatgaaaatgaagaagaatttcgagaggatgagcacgacgaccttagttcctcatacaaatctgacactgagactgaagacttgtcaggttagttatgttattgatattaatacaaatgcaaatatgtttatcgaagtgtt

SEQ ID NO: 28 - DNA, 201 bp, Cicer arietinum
  source 1..201: mol_type = genomic DNA
gaagacaatgatggctatgcttctgaaactgacgatgacaaccacaaatatgacgacgaatatgaaaatgaagaagaatttcgagaggatgagcacgacggccttagttcctcatacaaatctgacactgagactgaagacttgtcaggttagttatgttattgatattaatacaaatgcaaatatgtttatcgaagtgtt

SEQ ID NO: 29 - DNA, 201 bp, Cicer arietinum
  source 1..201: mol_type = genomic DNA
aaattttttaaaaataaaatgaatcaaattcaattataaaataattacaaaagacatttcattaaaatttaagttggcaattacatcattcgtgttaggcgtgggttccaaaaaccactatatttctatcaaaattctctttttatatatatcggatcatatctagagttgtaaaaaaatatagttaattgaatcaaatta

SEQ ID NO: 30 - DNA, 201 bp, Cicer arietinum
  source 1..201: mol_type = genomic DNA
aaattttttaaaaataaaatgaatcaaattcaattataaaataattacaaaagacatttcattaaaatttaagttggcaattacatcattcgtgttaggcatgggttccaaaaaccactatatttctatcaaaattctctttttatatatatcggatcatatctagagttgtaaaaaaatatagttaattgaatcaaatta

SEQ ID NO: 31 - DNA, 201 bp, Cicer arietinum
  source 1..201: mol_type = genomic DNA
cctcaaatttagacaaaggcttctctcgcagccacttcaccccaaaaaactgatggaacgaatctatggttatatttccatcagcagtagggcaggttgataccacagcatggtcaataaagtgacttctaacatgtttctttatgctaggctcacaacttatctcaacagctgcctgaaacaaaaaataaccatataatc

SEQ ID NO: 32 - DNA, 201 bp, Cicer arietinum
  source 1..201: mol_type = genomic DNA
cctcaaatttagacaaaggcttctctcgcagccacttcaccccaaaaaactgatggaacgaatctatggttatatttccatcagcagtagggcaggttgacaccacagcatggtcaataaagtgacttctaacatgtttctttatgctaggctcacaacttatctcaacagctgcctgaaacaaaaaataaccatataatc

SEQ ID NO: 33 - DNA, 201 bp, Cicer arietinum
  source 1..201: mol_type = genomic DNA
ttgtctgctatgtttgatggtgtctccaaatcattagtattgatgcatgatttgtccgtccgggctttaataacacggtatctggccataacagaagtctcgtcatttccagccgcctccttgtttgtgccagaaacagcataattccggatgaaactatacaagttttgacaaccccctgcttcaggtgtcaatgaatta

SEQ ID NO: 34 - DNA, 201 bp, Cicer arietinum
  source 1..201: mol_type = genomic DNA
ttgtctgctatgtttgatggtgtctccaaatcattagtattgatgcatgatttgtccgtccgggctttaataacacggtatctggccataacagaagtcttgtcatttccagccgcctccttgtttgtgccagaaacagcataattccggatgaaactatacaagttttgacaaccccctgcttcaggtgtcaatgaatta

(19) United States
(12) Patent Application Publication
(10) Pub. No.: US 2025/0351793 A1
(43) Pub. Date: Nov. 20, 2025

(54) HIGH PROTEIN LOW FAT CHICKPEA

(71) Applicant: EQUI-nom Ltd., Kibbutz Givat Brenner (IL)

(72) Inventors: Roi SILBERMAN, Rehovot (IL); Avichai AMRAD, Kfar Bilu (IL); Inbar RAFAEL, Tel Aviv (IL); Yoav SHOSHAN, Tel Aviv (IL); Menachem SKLARZ, Beer Sheva (IL); Oswald CRASTA, Lubbock, TX (US)

(73) Assignee: EQUI-nom Ltd., Kibbutz Givat Brenner (IL)

(21) Appl. No.: 18/866,648

(22) PCT Filed: Jun. 11, 2023
(86) PCT No.: PCT/IL2023/050597

§ 371 Date: Nov. 18, 2024

Related U.S. Application Data
(60) Provisional application No. 63/351,590, filed on Jun. 13, 2022.

(51) Int. Cl. A01H 6/54; A01H 5/10

(52) U.S. Cl. CPC A01H 6/54; A01H 5/10

(57) ABSTRACT

Chickpea plants with high protein content. low fat and/or modified protein composition are provided. as well as progeny or parts thereof. Phenotypic and genotypic analysis of many chickpea varieties were performed to derive markers for phenotypic traits that contribute to high protein content. low fat and/or modified protein composition. and a breeding simulation was used to identify the most common and most stable markers. Examples of such phenotypic traits include the protein content. the fat content as measured by near infrared spectroscopy. and protein composition and seed color traits. Following verification of trait stability over several generations. markers and marker cassettes comprising at least three specific QTLs were defined as being uniquely present in the developed chickpea lines. The resulting chickpea lines can be used to improve nutritional and organoleptic properties of various chickpea products and chickpea-based food ingredients. e.g., in plant-based protein products.

### SEQUENCE LISTING

A Sequence Listing conforming to the rules of WIPO Standard ST.26 is hereby incorporated by reference. Said Sequence Listing has been filed as an electronic document encoded as XML in UTF-8 text. The electronic document, created on Jun. 1, 2023, is entitled “10034-178WO1_ST26.xml”, and is 56,962 bytes in size.

### BACKGROUND OF THE INVENTION

### 1. Technical Field

The present invention relates to the field of chickpea breeding and products and, more particularly, to quantitative trait loci (QTLs, or QTL) associated with chickpea protein, chickpea protein composition, and/or chickpea fat content.

### 2. Discussion of Related Art

Chickpea (Cicer arietinum) is a pulse legume crop that is cultivated over a large range of soil and climate conditions, typically in subtropical climates, and is used for seeds, flour, and paste products such as humus and falafel, as well as a wide range of dishes. Additionally, chickpea products may be used as ingredients in plant-based protein products, e.g., in vegetarian food, such as meat, milk replacement products, and texturized vegetable products (TVPs).

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a chickpea plant, progeny thereof or part thereof, comprising a plurality of quantitative trait loci (QTLs) having a corresponding plurality of nucleic acid genetic markers that are associated with a plurality of phenotypic traits of seeds of the chickpea plant comprising at least one of a protein content trait, a fat content trait, a protein composition trait and a seed color trait, wherein the QTLs are genetic elements combined from different chickpea varieties by computationally supported breeding, wherein the QTLs comprise at least one of QTLs 1 to 17 with corresponding nucleic acid genetic markers set forth in SEQ ID NOs: 1 to 34. Seeds of the disclosed chickpea plants may have higher protein content and/or lower fat content.

These additional, and/or other aspects and/or advantages of the present invention are set forth in the detailed description which follows, possibly inferable from the detailed description, and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. In the accompanying drawings:

FIG. 1 is a high-level schematic illustration of seven of the eight chickpea chromosomes (2 n=16) with indications of the relevant quantitative trait loci, according to some embodiments of the invention.

FIGS. 2 and 3 provide comparisons of protein and fat levels, respectively, between chickpea lines having at least one of the five cassettes, according to some embodiments of the invention, and the leading large-seeded kabuli chickpea cultivar in Canada, CDC Orion (developed by the Crop Development Centre, University of Saskatchewan).

FIG. 4 illustrates the negative correlation in chickpea lines between protein and fat content in disclosed chickpea lines and the CDC Orion line, pooled together.

FIG. 5 is a high-level schematic illustration of a computationally supported breeding method according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing”, “deriving” or the like, may refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Chickpea plants with high protein content, low fat and/or modified protein composition, and parts thereof are provided. Phenotypic and genotypic analysis of many chickpea varieties were performed followed by analysis using Equi-nom Ltd. MANNA™ platform to derive markers for phenotypic traits that contribute to high protein content, low fat content and/or modified protein composition, and a breeding simulation was used to identify the most common and most stable phenotypic trait markers. Following verification of trait stability over several generations, QTL markers and QTL marker cassettes were defined as being uniquely present in the developed chickpea lines. Various food products, including meat substitutes made of the disclosed chickpea seeds have improved nutritional value, organoleptic properties and/or processing characteristics, and combinations thereof with plant-based protein products such as TVPs and/or meat/dairy replacements based on, e.g., cereals, other legumes and/or sesame improve the nutritional value, processing characteristics and/or taste of the combined product.

Advantageously, chickpea flour and chickpea protein concentrate or isolate contain higher protein levels, lower fat levels and/or improved protein composition. Therefore, chickpea can be used as a complementary supplement to cereal and legume proteins. For example, the disclosed chickpea variety products may be mixed with products from other legumes, sesame and/or cereal crops to yield products which have improved nutritional value and improved quantitative relations among nutritional elements. Moreover, mixing chickpea products with cereal/legume products may improve the nutritional value, processability and/or culinary traits.

Various embodiments comprise chickpea seeds, plants or part(s) thereof, that comprise a plurality of quantitative trait loci (QTLs) having a corresponding plurality of nucleic acid genetic markers that are associated with a plurality of phenotypic traits of the chickpea plant.

The QTLs are combined in the chickpea plants from a plurality of chickpea varieties according to computationally supported breeding tools. Phenotypic and genotypic analyses of many chickpea varieties were performed to derive markers for phenotypic traits that contribute to high protein, low fat and/or specified protein composition characteristics, and a breeding simulation was used to identify the most common and most stable markers. Examples of such phenotypic traits include the protein and fat content as measured by near infrared spectroscopy, as well as the levels of legumin and vicilin as protein components. Following verification of trait stability over several generations, QTL markers and QTL marker cassettes were defined as being uniquely present in the developed chickpea lines. The resulting chickpea lines can be used to increase chickpea protein quantity, composition and/or quality, as well as to improve the suitability of chickpea products as ingredient(s) in plant-based food, such as TVPs. Details concerning the QTL markers are provided in Table 1 below, and the methods used to develop and select the varieties are disclosed in FIG. 5.

It is noted that disclosed chickpea plants are hybridized in that none of the disclosed varieties occurs in nature or in known worldwide chickpea varieties. The herein provided chickpea plants are characterized by the disclosed QTL markers which were judiciously detected in other varieties, selected and gradually introduced in the disclosed combinations to yield the disclosed chickpea plants. Once specific disclosed chickpea plants were achieved, further breeding was used to stabilize the varieties and assure constant phenotypes for chickpea production, making the varieties pure lines. The term “hybridized” is used herein to define disclosed chickpea varieties having QTL markers and stable phenotypic traits, the chickpea varieties having been collected during the breeding process from different chickpea varieties that were determined and developed during the highly complicated computationally-supported breeding methods described below, in which the genotypes of multiple chickpea varieties have been judiciously combined and analyzed, to discover and accumulate the recited QTL markers and corresponding phenotypical traits into the disclosed chickpea plants. Although the recited chickpea plants are not genetically modified by sequences originating from other species, they cannot be reached merely by natural processes, as is evident by the detailed and intentional breeding program that was applied to specifically measure required characteristics, detect corresponding markers using bioinformatics methods and combine the detected QTLs in the selected varieties by classic breeding approaches (e.g., hand pollination crosses and single plant selections). For example, any further generation derived from the disclosed chickpea plants with specific characteristics (e.g., high protein, low fat and/or specified protein composition) is understood to have similar characteristics (unless these were intentionally bred out of the lines).

As described herein, five unique combinations of QTLs, referred to as QTL cassettes, were detected to differentiate disclosed chickpea varieties from worldwide chickpea lines. The cassettes' discovery was based on ca. 276 elite chickpea lines and a set of ca. 4055 world accessions. Following the cassettes' discovery, the disclosed chickpea varieties germplasm was tested with respect to 4055 world accessions to demonstrate their uniqueness, as being clearly distinct from known chickpea varieties (see Table 3).

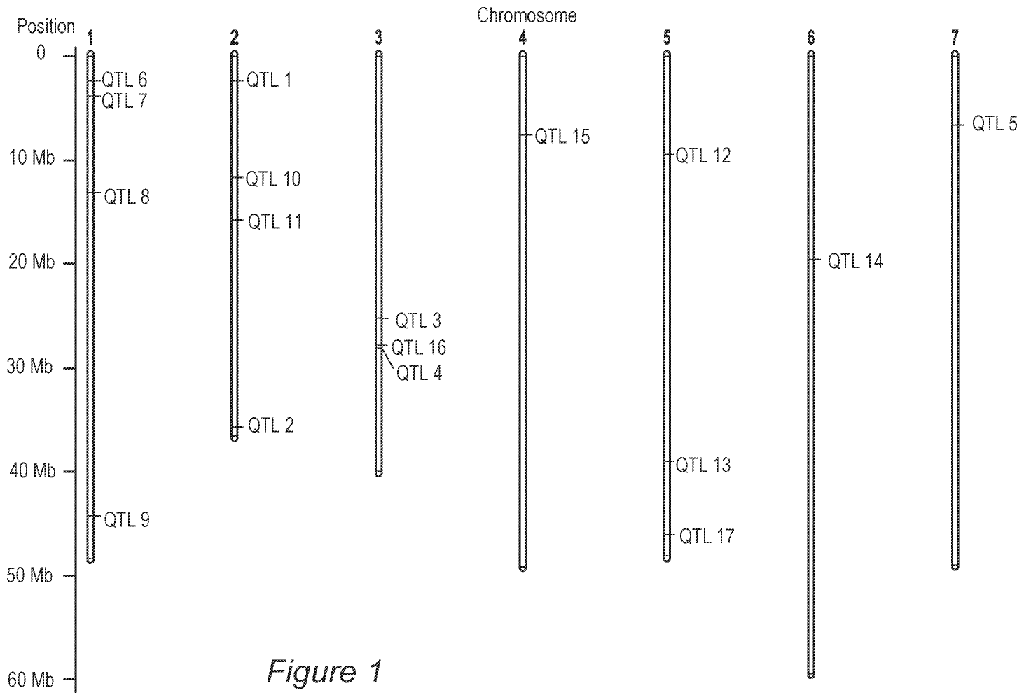

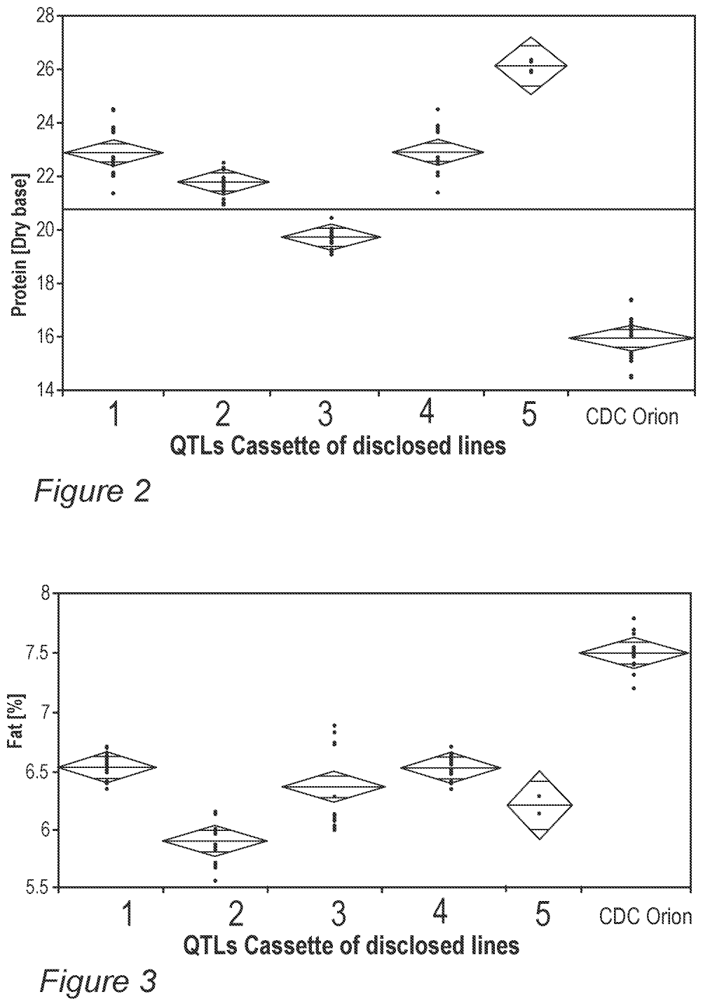

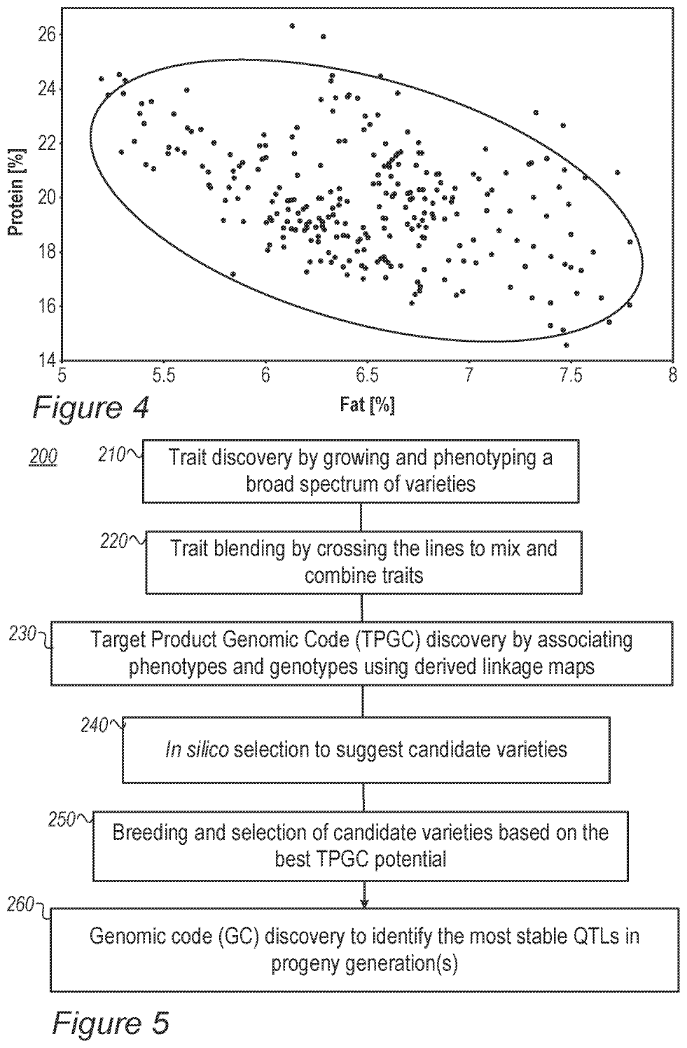

FIG. 1 is a high-level schematic illustration of seven of the eight chickpea chromosomes (2 n=16) with indications of the relevant markers' loci, according to some embodiments of the invention. It is noted that chromosome 8 does not include any marker. Table 1 below lists the QTL markers, their combinations into cassettes and their respective identified phenotypic traits. The chromosomal position refers to the Genome version ASM33114v1 (Accession GCF_000331145.1 in RefSeq database). FIGS. 2 and 3 provide comparisons of protein and fat levels, respectively. between lines having at least one of the five cassettes, according to some embodiments of the invention, and the leading large-seeded kabuli chickpea cultivar in Canada, CDC Orion (developed by the Crop Development Centre, University of Saskatchewan). FIG. 4 illustrates the negative correlation in chickpea lines between protein and fat content, in disclosed chickpea lines and the CDC Orion line, pooled together. The data is based on growing trials carried out in Washington state in 2021. Table 2 summarizes the phenotypic effects improved by the disclosed chickpea lines.

Protein content was measured by using Perten Instruments DA 7200 NIR analysis system, calibrated for chickpea seeds. Protein content was measured as grams (“gr”) of protein per 100 gr chickpea seeds (percent as-is, without drying).

Protein composition (e.g., total legumin content, % fractions of vicilin 2, vicilin 5 and legumin) was analyzed by a non-reducing sodium dodecyl sulphate polyacrylamide gel electrophoresis (SDS-PAGE) using a Criterion™ Vertical Electrophoresis Cell (BioRad Laboratories, Herculas, California, USA). Samples were prepared by mixing 6 μL of sample (1 g of flour from ground chickpea seeds/40 ml NaOH, 3 mM) with 34 μL of sample buffer solution (24 μl of distilled water, 9 μl of 4X Laemmli sample buffer and 1 μl of β-mercaptoethanol). Samples were boiled for 5 min at 100° C. and then 10 μL of each sample were loaded on a 12% Criterion™ TGX™ Precast Gel (Bio-Rad Laboratories, Inc. Hercules, USA) and separated at 110 Volt for 90 min. Afterwards, samples were stained with InstantBlue® Coomassie Protein Stain (Abcam) and rinsed with distilled water. Protein detection, analysis and documentation was performed by an imaging system (gel Doc™ EZ Imager, Bio-Rad).

Fat content was measured in the near infrared spectrum of ca. 500 chickpea seeds (bulk) per line after harvesting using Perten Instruments DA 7200 NIR analysis system, calibrated for chickpea seeds.

Seed color was estimated according to a Lab chart standard scale measured by VIBE QM3 seed analyzer.

TABLE 1
QTL markers, their combinations into cassettes and
their respective identified phenotypic traits.

| | SEQ | Chromosome- | | | QTL | | | |
|---|---|---|---|---|---|---|---|---|
| QTL | ID NO: | Position | | | P-value | Phenotypic Trait | | |
| 1 | 1, 2 | Chr 2-3965579 | | | <0.0001 | Protein % | | |
| 2 | 3, 4 | Chr 2-35660578 | | | <0.0001 | Vicilin 5 fraction (%) | | |
| 3 | 5, 6 | Chr 3-25247714 | | | <0.0001 | Vicilin 5 fraction (%), | | |
| | | | | | | Vicilin 2 fraction (%), | | |
| | | | | | | Legumin total (%)/Vicilin | | |
| | | | | | | total (%) | | |
| 4 | 7, 8 | Chr 3-28079832 | | | <0.0001 | Total legumin | | |
| 5 | 9, 10 | Chr 7-6650941 | | | 0.0002 | Seed color | | |
| 6 | 11, 12 | Chr 1-2338298 | | | <0.0001 | NIR Fat | | |
| 7 | 13, 14 | Chr 1-3856206 | | | <0.0001 | NIR Fat | | |
| 8 | 15, 16 | Chr 1-13087254 | | | <0.0001 | Protein % | | |
| 9 | 17, 18 | Chr 1-44225557 | | | <0.0001 | Protein % | | |
| 10 | 19, 20 | Chr 2-11606490 | | | <0.0001 | Protein % | | |
| 11 | 21, 22 | Chr 2-15744378 | | | <0.0001 | Protein % | | |
| 12 | 23, 24 | Chr 5-9482409 | | | <0.0001 | Protein % | | |
| 13 | 25, 26 | Chr 5-38996078 | | | <0.0001 | NIR Fat | | |
| 14 | 27, 28 | Chr 6-19487308 | | | <0.0001 | Protein % | | |
| 15 | 29, 30 | Chr 4-7543422 | | | <0.0001 | Protein % | | |
| 16 | 31, 32 | Chr 3-27890849 | | | <0.0001 | Protein % | | |
| 17 | 33, 34 | Chr 5-46143627 | | | <0.0001 | Total legumin | | |
| | | | | Cassettes | | | | |
| | SEQ | Allele | | (with Respective QTLs) | | | | |
| QTL | ID NO: | 1 | 2 | 1 | 2 | 3 | 4 | 5 |
| 1 | 1, 2 | A | G | AA/AG | | | | |
| 2 | 3, 4 | C | G | CC/CG | | | CC/CG | |
| 3 | 5, 6 | A | G | AA/AG | | | AA/AG | GG/GA |
| 4 | 7, 8 | T | C | TT/TC | | | | |
| 5 | 9, 10 | C | G | CC/CG | | | | |
| 6 | 11, 12 | T | C | | TT/TC | | | |
| 7 | 13, 14 | T | C | | TT/TC | TT/TC | | |
| 8 | 15, 16 | G | C | | GG/GC | | | |
| 9 | 17, 18 | C | G | | CC/CG | | | |
| 10 | 19, 20 | C | T | | CC/CT | | | |
| 11 | 21, 22 | T | C | | TT/TC | | | |
| 12 | 23, 24 | T | C | | TT/TC | | | TT/TC |
| 13 | 25, 26 | G | C | | GG/GC | CC/CG | GG/GC | GG/GC |
| 14 | 27, 28 | A | G | | AA/AG | | | |
| 15 | 29, 30 | G | A | | | GG/GA | | AA/AG |
| 16 | 31, 32 | T | C | | | | | TT/TC |
| 17 | 33, 34 | C | T | | | | CC/CT | |

Disclosed chickpea plants were derived by computationally supported breeding methods to yield plants which are different and distinct from any prior art chickpea varieties. Specifically, disclosed chickpea plants are grouped herein by combinations of QTLs denoted in Table 1 as cassettes 1, 2, 3, 4 and 5.

FIG. 2 provides a comparison of protein level between chickpea lines having at least one of the five cassettes, according to some embodiments of the invention, and the CDC Orion cultivar. Lines having any one of the five cassettes clearly and significantly have higher protein levels, e.g., typically having between 19% and 26% protein compared to the CDC Orion cultivar having around 16% protein, dry base values, the difference being significant with p<0.0001.

FIG. 3 provides a comparison of fat level between lines having at least one of the five cassettes, according to some embodiments of the invention, and the CDC Orion cultivar. Lines having any one of the five cassettes clearly and significantly have lower fat levels, e.g., typically between 5.5% and 7% compared to the CDC Orion cultivar having around 7.5% fat, the difference being significant with p<0.0001.

FIG. 4 illustrates the negative correlation in chickpea lines between protein and fat content, in disclosed and CDC Orion cultivar line. The regression coefficient is −0.44 with p<0.0001. FIG. 4 includes data for disclosed as well as CDC Orion cultivar chickpea lines.

Table 2 provides the effects and improved performance of some of the disclosed chickpea varieties with different cassettes with respect to the protein and fat oil content, compared to the CDC Orion line not expressing any of the cassettes.

TABLE 2
Protein and fat content in chickpea lines with the cassettes
and comparison to CDC Orion line lacking the cassettes.

| Cassette/ | Average | Average | Compared to CDC Orion | |
|---|---|---|---|---|
| Variety | Protein (%) | Fat (%) | Protein | Fat |
| Cassette 1 | 22.89 | 6.52 | +43.6% | −12.7% |
| Cassette 2 | 21.84 | 5.92 | +37.1% | −20.8% |
| Cassette 3 | 18.97 | 6.44 | +19.1% | −13.8% |
| Cassette 4 | 22.89 | 6.52 | +43.6% | −12.7% |
| Cassette 5 | 26.12 | 6.21 | +63.9% | −17.0% |
| CDC Orion | 15.93 | 7.48 | 0 | 0 |

Additional tests comparing disclosed varieties with additional worlds varieties indicate similar results and will be finalized in the current growing season.

Disclosed chickpea plants provide increases of protein content of at least 20%, reduction of fat content by at least 10% and/or modifications of the protein composition with respect to chickpea varieties that do not include the disclosed cassettes (CDC Orion cultivar). Correspondingly, the term “high protein” refers to an increase in protein content of at least 20% above the CDC Orion cultivar, and the term “low fat” refers to a decrease in fat content of at least 10% below the CDC Orion cultivar. In certain embodiments, disclosed chickpea plants provide increases of protein content by about 40%, reduction of fat content by about 20% and/or modifications of the protein composition with respect to chickpea varieties that do not include the disclosed cassettes (CDC Orion cultivar).

In various embodiments, certain disclosed varieties increase the protein content by any of at least 10%, at least 15%, at least 20%, at least 25%, at least 30%, at least 35%, at least 40%, at least 45%, at least 50%, at least 55%, at least 60% or at least 65% with respect to chickpea varieties that do not include the disclosed cassettes (CDC Orion cultivar). In some embodiments, certain disclosed varieties decrease the fat content by any of at least 5%, at least 10%, at least 15% or at least 20% with respect to chickpea varieties that do not include the disclosed cassettes (CDC Orion cultivar). In various embodiments, certain disclosed varieties modify the protein composition (e.g., have modified total legumin content, % fractions of vicilin 2, vicilin 5 and legumin, etc.) with respect to current varieties (CDC Orion cultivar).

Advantageously, disclosed embodiments provide chickpea plants with high protein, low fat and/or favorable protein composition that may improve the nutritional value of the chickpea seeds and/or enable or improve using chickpea products and ingredients, e.g., in meat and dairy replacements. These varieties and products therefrom (e.g., isolates and/or concentrates) may be used to enrich products made from most legume and/or cereal crops, such as pea, soybean, wheat, barley, rice, corn and others that are low in protein and/or have inferior processing or organoleptic properties. Accordingly, the disclosed chickpea plants and products thereof may be used as complementary supplements to cereal and other-legume proteins.

QTL 1, as used herein, refers to a polymorphic genetic locus linked to a genetic marker at position 3965579 on chickpea chromosome 2. The two alleles of the genetic marker at QTL 1 have the SNP bases “A” or “G”, as set forth respectively in the nucleic acid sequences of SEQ ID NOs: 1 and 2. In cassette 1, QTL 1 is homozygous for allele “A” (SEQ ID NO: 1) or heterozygous (includes both SEQ ID NOs: 1 and 2).

SEQ ID NO: 1 (SNP base bold):
ATACCTTTGGGTTTTAACCAATAACTTTTATTCATTAATTTTGCAGTGGCCTCTTTCAG
TCATTGAATCACACAGCGACAAGGTATAAATCTTTCTTCCC A TGTTTTATAAGGCGA
AGTAAACAGCGTTGCTTGCTAAACAGCGGTGTAGTGCAATTTGAACAAACCATTATT
ATTTTGCAATACATAATTTTGTATAAAG
SEQ ID NO: 2 (SNP base bold):
ATACCTTTGGGTTTTAACCAATAACTTTTATTCATTAATTTTGCAGTGGCCTCTTTCAG
TCATTGAATCACACAGCGACAAGGTATAAATCTTTCTTCCC G TGTTTTATAAGGCGA
AGTAAACAGCGTTGCTTGCTAAACAGCGGTGTAGTGCAATTTGAACAAACCATTATT
ATTTTGCAATACATAATTTTGTATAAAG

QTL 2, as used herein, refers to a polymorphic genetic locus linked to a genetic marker at position 35660578 on chickpea chromosome 2. The two alleles of the genetic marker at QTL 2 have the SNP bases “C” or “G”, as set forth respectively in the nucleic acid sequences of SEQ ID NOs: 3 and 4. In cassettes 1 and 4, QTL 2 is homozygous for allele “C” (SEQ ID NO: 3) or heterozygous (includes both SEQ ID NOs: 3 and 4).

SEQ ID NO: 3 (SNP base bold):
CACAGGCACTTGGTTTTCTTAGTGACTTCAAATTGGGACACTACATGAAAATTCCTCC
AAAAGCTATGTTCCTAGTACAGGTAAGAATGTAATAATGGAT C CATAGTTTGCCAA
ATATCTAACTCGAAAGCAATACAACATATCTATTGGGTATGGCTCATGGTTTATTGAT
AAATGTAATTCAAAGCCACCCGTGGTACT
SEQ ID NO: 4 (SNP base bold):
CACAGGCACTTGGTTTTCTTAGTGACTTCAAATTGGGACACTACATGAAAATTCCTCC
AAAAGCTATGTTCCTAGTACAGGTAAGAATGTAATAATGGAT G CATAGTTTGCCAA
ATATCTAACTCGAAAGCAATACAACATATCTATTGGGTATGGCTCATGGTTTATTGAT
AAATGTAATTCAAAGCCACCCGTGGTACT

QTL 3, as used herein, refers to a polymorphic genetic locus linked to a genetic marker at position 25247714 on chickpea chromosome 3. The two alleles of the genetic marker at QTL 3 have the SNP bases “A” or “G”, as set forth respectively in the nucleic acid sequences of SEQ ID NOs: 5 and 6. In cassettes 1 and 4, QTL 3 is homozygous for allele “A” (SEQ ID NO: 5) or heterozygous (includes both SEQ ID NOs: 5 and 6). In cassette 5, QTL 3 is homozygous for allele “G” (SEQ ID NO: 6) or heterozygous (includes both SEQ ID NOs: 5 and 6).

SEQ ID NO: 5 (SNP base bold):
TTTAGTTTTCTTATGTTGTGGCAAGTCTCAAATGATTTGTAGATC
ACTTTTGTGTTTTTGTGGCAATGTCCAATCTATATGCCACAATAA
GGTGTATTTG A TGAAAGCAGAACTCTCGTACCTTTTATAAGAT
TTCTTTTGCATATCAGTGAACTTGGGATAAGATCGGGCAATTGGC
ATCTATTTGGTGCAAATGTCATA
SEQ ID NO: 6 (SNP base bold):
TTTAGTTTTCTTATGTTGTGGCAAGTCTCAAATGATTTGTAGATC
ACTTTTGTGTTTTTGTGGCAATGTCCAATCTATATGCCACAATAA
GGTGTATTTG G TGAAAGCAGAACTCTCGTACCTTTTATAAGAT
TTCTTTTGCATATCAGTGAACTTGGGATAAGATCGGGCAATTGGC
ATCTATTTGGTGCAAATGTCATA

QTL 4, as used herein, refers to a polymorphic genetic locus linked to a genetic marker at position 28079832 on chickpea chromosome 3. The two alleles of the genetic marker at QTL 4 have the SNP bases “T” or “C”, as set forth respectively in the nucleic acid sequences of SEQ ID NOs: 7 and 8. In cassette 1, QTL 4 is homozygous for allele “T” (SEQ ID NO: 7) or heterozygous (includes both SEQ ID NOs: 7 and 8).

SEQ ID NO: 7 (SNP base bold):
ATCAACATCACCATCTCCAACCTCTTTGGGATCAACTAGTACCGA
AACCACCATTGAAGATTTATTACCAGGGAGGTGGCCTTTCGGCAA
ATTTCTAGCA T GCTCTTCAGTTATGTTCAGAGTGGATCCAGGA
AGTGGATCTGGGAGCGTGTGGAGGATTCTTCTGTTCCTGCTCTTG
TTGACAGTGGTCGCATTACGACG
SEQ ID NO: 8 (SNP base bold):
ATCAACATCACCATCTCCAACCTCTTTGGGATCAACTAGTACCGA
AACCACCATTGAAGATTTATTACCAGGGAGGTGGCCTTTCGGCAA
ATTTCTAGCA C GCTCTTCAGTTATGTTCAGAGTGGATCCAGGA
AGTGGATCTGGGAGCGTGTGGAGGATTCTTCTGTTCCTGCTCTTG
TTGACAGTGGTCGCATTACGACG

QTL 5, as used herein, refers to a polymorphic genetic locus linked to a genetic marker at position 6650941 on chickpea chromosome 7. The two alleles of the genetic marker at QTL 5 have the SNP bases “C” or “G”, as set forth respectively in the nucleic acid sequences of SEQ ID NOs: 9 and 10. In cassette 1, QTL 5 is homozygous for allele “C” (SEQ ID NO: 9) or heterozygous (includes both SEQ ID NOs: 9 and 10).

SEQ ID NO: 9 (SNP base bold):
ACTTGAATTTCTCTTGGTAATGCTCACTTGTCATAATAACCATTG
ATAATGGAATGCACATTTTCATCGTTGTTGCTTTTGTAACGAGTT
AATGACATAA C TTAGAAGGATTTTTCCCAGGGATGACTGAATC
GAAAGTTTAATGGTCAGTATTATGGTTTTGGATGGTTTAATAGTT
CTTCTGGAATTTCAATTAGCTTG
SEQ ID NO: 10 (SNP base bold):
ACTTGAATTTCTCTTGGTAATGCTCACTTGTCATAATAACCATTG
ATAATGGAATGCACATTTTCATCGTTGTTGCTTTTGTAACGAGTT
AATGACATAA G TTAGAAGGATTTTTCCCAGGGATGACTGAATC
GAAAGTTTAATGGTCAGTATTATGGTTTTGGATGGTTTAATAGTT
CTTCTGGAATTTCAATTAGCTTG

QTL 6, as used herein, refers to a polymorphic genetic locus linked to a genetic marker at position 2338298 on chickpea chromosome 1. The two alleles of the genetic marker at QTL 6 have the SNP bases “T” or “C”, as set forth respectively in the nucleic acid sequences of SEQ ID NOS: 11 and 12. In cassette 2, QTL 6 is homozygous for allele “T” (SEQ ID NO: 11) or heterozygous (includes both SEQ ID NOs: 11 and 12).

SEQ ID NO: 11 (SNP base bold):
TGCAGGTAATAACAGTCTTGTGCATTTTTCATTGATCCTGTTATT
GGTCGTGTGTCTAACATTAGATGTTTTTATTTCTTCCAGAGATAC
GGTCCTCCAC T GTCATACCCTCATCTGAAAATCCCTGGACTGA
ATGCTCCCATTCCCACTGGAGCTAGCTTTGGTTATCATCCTGGCG
GATGGGGAAAACCTCCAGTTGAC
SEQ ID NO: 12 (SNP base bold):
TGCAGGTAATAACAGTCTTGTGCATTTTTCATTGATCCTGTTATT
GGTCGTGTGTCTAACATTAGATGTTTTTATTTCTTCCAGAGATAC
GGTCCTCCAC C GTCATACCCTCATCTGAAAATCCCTGGACTGA
ATGCTCCCATTCCCACTGGAGCTAGCTTTGGTTATCATCCTGGCG
GATGGGGAAAACCTCCAGTTGAC

QTL 7, as used herein, refers to a polymorphic genetic locus linked to a genetic marker at position 3856206 on chickpea chromosome 1. The two alleles of the genetic marker at QTL 7 have the SNP bases “T” or “C”, as set forth respectively in the nucleic acid sequences of SEQ ID NOs: 13 and 14. In cassettes 2 and 3, QTL 7 is homozygous for allele “T” (SEQ ID NO: 13) or heterozygous (includes both SEQ ID NOs: 13 and 14).

SEQ ID NO: 13 (SNP base bold):
AACAATCAAAGTTCTAGGACACAATAAAAAACATAAAAGACCAAA
GATTAGGAAGGAATAGTTCATATCAAAACGATTTGGACTGCAACA
TCAACTTATT T TAAGTCTCTTCAACCGCGATTCAGGCCACATC
ATTCACACATATTACTACGATTCTATGCAATATTAAAGTTCGCAA
CACAGCCACAATTTAAAGCTATG
SEQ ID NO: 14 (SNP base bold):
AACAATCAAAGTTCTAGGACACAATAAAAAACATAAAAGACCAAA
GATTAGGAAGGAATAGTTCATATCAAAACGATTTGGACTGCAACA
TCAACTTATT C TAAGTCTCTTCAACCGCGATTCAGGCCACATC
ATTCACACATATTACTACGATTCTATGCAATATTAAAGTTCGCAA
CACAGCCACAATTTAAAGCTATG

QTL 8, as used herein, refers to a polymorphic genetic locus linked to a genetic marker at position 13087254 on chickpea chromosome 1. The two alleles of the genetic marker at QTL 8 have the SNP bases “G” or “C”, as set forth respectively in the nucleic acid sequences of SEQ ID NOs: 15 and 16. In cassette 2, QTL 8 is homozygous for allele “G” (SEQ ID NO: 15) or heterozygous (includes both SEQ ID NOs: 15 and 16).

SEQ ID NO: 15 (SNP base bold):
CCAAAAGAATTGATTTTTTATTTTGGCGGTGGTGTTTTTGTTATG
CAGTTTTTAAGTACAACAAAGGATCGGATTCAGTGTTGTTGGTAA
ACAAACAAGA G TATGATTCATGCAACACAAAGAATCCAATTAC
CAAGATGGACGGTGGAGATTCAATTTTCACTTTGGATAAATCAGG
TCCATTTTTCTTCATTAGTGGCA
SEQ ID NO: 16 (SNP base bold):
CCAAAAGAATTGATTTTTTATTTTGGCGGTGGTGTTTTTGTTATG
CAGTTTTTAAGTACAACAAAGGATCGGATTCAGTGTTGTTGGTAA
ACAAACAAGA C TATGATTCATGCAACACAAAGAATCCAATTAC
CAAGATGGACGGTGGAGATTCAATTTTCACTTTGGATAAATCAGG
TCCATTTTTCTTCATTAGTGGCA

QTL 9, as used herein, refers to a polymorphic genetic locus linked to a genetic marker at position 44225557 on chickpea chromosome 1. The two alleles of the genetic marker at QTL 9 have the SNP bases “C” or “G”, as set forth respectively in the nucleic acid sequences of SEQ ID NOs: 17 and 18. In cassette 2, QTL 8 is homozygous for allele “C” (SEQ ID NO: 17) or heterozygous (includes both SEQ ID NOs: 17 and 18).

SEQ ID NO: 17 (SNP base bold):
GTAAGAAAAGTGGGAGATAGTTAATTAAGTAAAGTGTGACACCAA
GTTGCGCCATATTTGTGAAGGAATGGAGCCAGAGATTTATAAAGT
GGTTGGGATA C AGAAAGTAGAAGAAAGGTGTTGGACTTGGAAC
AAGATTTATTTTGTTATAAAAAACACCAACAAGATTTAAAACTTA
TCTAACCCTTAGGTTGAATTGTG
SEQ ID NO: 18 (SNP base bold):
GTAAGAAAAGTGGGAGATAGTTAATTAAGTAAAGTGTGACACCAA
GTTGCGCCATATTTGTGAAGGAATGGAGCCAGAGATTTATAAAGT
GGTTGGGATA G AGAAAGTAGAAGAAAGGTGTTGGACTTGGAAC
AAGATTTATTTTGTTATAAAAAACACCAACAAGATTTAAAACTTA
TCTAACCCTTAGGTTGAATTGTG

QTL 10, as used herein, refers to a polymorphic genetic locus linked to a genetic marker at position 11606490 on chickpea chromosome 2. The two alleles of the genetic marker at QTL 10 have the SNP bases “C” or “T”, as set forth respectively in the nucleic acid sequences of SEQ ID NOs: 19 and 20. In cassette 2, QTL 10 is homozygous for allele “C” (SEQ ID NO: 19) or heterozygous (includes both SEQ ID NOs: 19 and 20).

SEQ ID NO: 19 (SNP base bold):
TCAATGCATATAAATTAAATTCTAACGAGAATACTTATCAGTTAT
CACTATTAGTGCTTTAATCTCACTATTGAACTTAGTTATTTGATC
TCCAAAGGGC C TGTGTAACTTACACTTTCTGAAAATTGTGGAC
TAGGAATTCCTGCAGCTAAGGATCTAAACAGAGGGGTAACTTGAA
GAGGGGAACTGAACTCTGTACTT
SEQ ID NO: 20 (SNP base bold):
TCAATGCATATAAATTAAATTCTAACGAGAATACTTATCAGTTAT
CACTATTAGTGCTTTAATCTCACTATTGAACTTAGTTATTTGATC
TCCAAAGGGC T TGTGTAACTTACACTTTCTGAAAATTGTGGAC
TAGGAATTCCTGCAGCTAAGGATCTAAACAGAGGGGTAACTTGAA
GAGGGGAACTGAACTCTGTACTT

QTL 11, as used herein, refers to a polymorphic genetic locus linked to a genetic marker at position 15744378 on chickpea chromosome 2. The two alleles of the genetic marker at QTL 11 have the SNP bases “T” or “C”, as set forth respectively in the nucleic acid sequences of SEQ ID NOs: 21 and 22. In cassette 2, QTL 11 is homozygous for allele “T” (SEQ ID NO: 21) or heterozygous (includes both SEQ ID NOs: 21 and 22).

SEQ ID NO: 21 (SNP base bold):
TGTCAAATCTTGATCATAAATTTTCCTTTTTAGTTGTGACACATA
CCACTTAGAGTTACTTCTAAGAACGCATATCATCTGATACTTCAC
TTGTATCAGC T GACGACATCTATGGATGTAGACGTTGACGTAA
AGACACAAACATGAGTTTTTTTTGTCAAACCAAGTATCTTCTGAT
CTTCAGTAACCGAATGATGTTAA
SEQ ID NO: 22 (SNP base bold):
TGTCAAATCTTGATCATAAATTTTCCTTTTTAGTTGTGACACATA
CCACTTAGAGTTACTTCTAAGAACGCATATCATCTGATACTTCAC
TTGTATCAGC C GACGACATCTATGGATGTAGACGTTGACGTAA
AGACACAAACATGAGTTTTTTTTGTCAAACCAAGTATCTTCTGAT
CTTCAGTAACCGAATGATGTTAA

QTL 12, as used herein, refers to a polymorphic genetic locus linked to a genetic marker at position 9482409 on chickpea chromosome 5. The two alleles of the genetic marker at QTL 12 have the SNP bases “T” or “C”, as set forth respectively in the nucleic acid sequences of SEQ ID NOs: 23 and 24. In cassettes 2 and 5, QTL 12 is homozygous for allele “T” (SEQ ID NO: 23) or heterozygous (includes both SEQ ID NOs: 23 and 24).

SEQ ID NO: 23 (SNP base bold):
AGATTATAATTTTTAATATTGAAAAAAATATTAAATATATTGAAT
GTATGCATGTATATAAAATTAAAAAATGACAAGAATATTAGTAGC
GGAACAAGTT T GGACGCAGGTGAATATCAATACACCAAAATCC
GTCTCTACTCCTAAGTTTTAATTTCGAAAAAAATTCATACGCAAT
CAAAATAAATTTCTCCATCTAAA
SEQ ID NO: 24 (SNP base bold):
AGATTATAATTTTTAATATTGAAAAAAATATTAAATATATTGAAT
GTATGCATGTATATAAAATTAAAAAATGACAAGAATATTAGTAGC
GGAACAAGTT C GGACGCAGGTGAATATCAATACACCAAAATCC
GTCTCTACTCCTAAGTTTTAATTTCGAAAAAAATTCATACGCAAT
CAAAATAAATTTCTCCATCTAAA

QTL 13, as used herein, refers to a polymorphic genetic locus linked to a genetic marker at position 38996078 on chickpea chromosome 5. The two alleles of the genetic marker at QTL 13 have the SNP bases “G” or “C”, as set forth respectively in the nucleic acid sequences of SEQ ID NOs: 25 and 26. In cassettes 2, 4 and 5, QTL 13 is homozygous for allele “G” (SEQ ID NO: 25) or heterozygous (includes both SEQ ID NOs: 25 and 26). In cassette 3, QTL 13 is homozygous for allele “C” (SEQ ID NO: 26) or heterozygous (includes both SEQ ID NOs: 25 and 26).

SEQ ID NO: 25 (SNP base bold):
CACCTGTTATTGATGTTACTCAATTTGGTTTCTTCAAGCTTTTGG
GTAAAGGTGTTTTGCCTCAGAATCAGCCATTTGTTGTGAAGACTA
AACTCATTTC G AAGATCGCTGAGAAGAAGATTAAGGAAGCCGG
TGGTGCTGTTGTTCTTACCGCTTAATTTTT
SEQ ID NO: 26 (SNP base bold):
CACCTGTTATTGATGTTACTCAATTTGGTTTCTTCAAGCTTTTGG
GTAAAGGTGTTTTGCCTCAGAATCAGCCATTTGTTGTGAAGACTA
AACTCATTTC C AAGATCGCTGAGAAGAAGATTAAGGAAGCCGG
TGGTGCTGTTGTTCTTACCGCTTAATTTTT

QTL 14, as used herein, refers to a polymorphic genetic locus linked to a genetic marker at position 19487308 on chickpea chromosome 6. The two alleles of the genetic marker at QTL 14 have the SNP bases “A” or “G”, as set forth respectively in the nucleic acid sequences of SEQ ID NOs: 27 and 28. In cassette 2, QTL 14 is homozygous for allele “A” (SEQ ID NO: 27) or heterozygous (includes both SEQ ID NOs: 27 and 28).

SEQ ID NO: 27 (SNP base bold):
GAAGACAATGATGGCTATGCTTCTGAAACTGACGATGACAACCAC
AAATATGACGACGAATATGAAAATGAAGAAGAATTTCGAGAGGAT
GAGCACGACG A CCTTAGTTCCTCATACAAATCTGACACTGAGA
CTGAAGACTTGTCAGGTTAGTTATGTTATTGATATTAATACAAAT
GCAAATATGTTTATCGAAGTGTT
SEQ ID NO: 28 (SNP base bold):
GAAGACAATGATGGCTATGCTTCTGAAACTGACGATGACAACCAC
AAATATGACGACGAATATGAAAATGAAGAAGAATTTCGAGAGGAT
GAGCACGACG G CCTTAGTTCCTCATACAAATCTGACACTGAGA
CTGAAGACTTGTCAGGTTAGTTATGTTATTGATATTAATACAAAT
GCAAATATGTTTATCGAAGTGTT

QTL 15, as used herein, refers to a polymorphic genetic locus linked to a genetic marker at position 7543422 on chickpea chromosome 4. The two alleles of the genetic marker at QTL 15 have the SNP bases “G” or “A”, as set forth respectively in the nucleic acid sequences of SEQ ID NOs: 29 and 30. In cassette 3, QTL 15 is homozygous for allele “G” (SEQ ID NO: 29) or heterozygous (includes both SEQ ID NOs: 29 and 30). In cassette 5, QTL 15 is homozygous for allele “A” (SEQ ID NO: 30) or heterozygous (includes both SEQ ID NOs: 29 and 30).

SEQ ID NO: 29 (SNP base bold):
AAATTTTTTAAAAATAAAATGAATCAAATTCAATTATAAAATAAT
TACAAAAGACATTTCATTAAAATTTAAGTTGGCAATTACATCATT
CGTGTTAGGC G TGGGTTCCAAAAACCACTATATTTCTATCAAA
ATTCTCTTTTTATATATATCGGATCATATCTAGAGTTGTAAAAAA
ATATAGTTAATTGAATCAAATTA
SEQ ID NO: 30 (SNP base bold):
AAATTTTTTAAAAATAAAATGAATCAAATTCAATTATAAAATAAT
TACAAAAGACATTTCATTAAAATTTAAGTTGGCAATTACATCATT
CGTGTTAGGC A TGGGTTCCAAAAACCACTATATTTCTATCAAA
ATTCTCTTTTTATATATATCGGATCATATCTAGAGTTGTAAAAAA
ATATAGTTAATTGAATCAAATTA

QTL 16, as used herein, refers to a polymorphic genetic locus linked to a genetic marker at position 27890849 on chickpea chromosome 3. The two alleles of the genetic marker at QTL 16 have the SNP bases “T” or “C”, as set forth respectively in the nucleic acid sequences of SEQ ID NOs: 31 and 32. In cassette 5, QTL 16 is homozygous for allele “T” (SEQ ID NO: 31) or heterozygous (includes both SEQ ID NOs: 31 and 32).

SEQ ID NO: 31 (SNP base bold):
CCTCAAATTTAGACAAAGGCTTCTCTCGCAGCCACTTCACCCCAA
AAAACTGATGGAACGAATCTATGGTTATATTTCCATCAGCAGTAG
GGCAGGTTGA T ACCACAGCATGGTCAATAAAGTGACTTCTAAC
ATGTTTCTTTATGCTAGGCTCACAACTTATCTCAACAGCTGCCTG
AAACAAAAAATAACCATATAATC
SEQ ID NO: 32 (SNP base bold):
CCTCAAATTTAGACAAAGGCTTCTCTCGCAGCCACTTCACCCCAA
AAAACTGATGGAACGAATCTATGGTTATATTTCCATCAGCAGTAG
GGCAGGTTGA C ACCACAGCATGGTCAATAAAGTGACTTCTAAC
ATGTTTCTTTATGCTAGGCTCACAACTTATCTCAACAGCTGCCTG
AAACAAAAAATAACCATATAATC

QTL 17, as used herein, refers to a polymorphic genetic locus linked to a genetic marker at position 46143627 on chickpea chromosome 5. The two alleles of the genetic marker at QTL 17 have the SNP bases “C” or “T”, as set forth respectively in the nucleic acid sequences of SEQ ID NOs: 33 and 34. In cassette 4, QTL 17 is homozygous for allele “C” (SEQ ID NO: 33) or heterozygous (includes both SEQ ID NOs: 33 and 34).

SEQ ID NO: 33 (SNP base bold):
TTGTCTGCTATGTTTGATGGTGTCTCCAAATCATTAGTATTGATG
CATGATTTGTCCGTCCGGGCTTTAATAACACGGTATCTGGCCATA
ACAGAAGTCT C GTCATTTCCAGCCGCCTCCTTGTTTGTGCCAG
AAACAGCATAATTCCGGATGAAACTATACAAGTTTTGACAACCCC
CTGCTTCAGGTGTCAATGAATTA
SEQ ID NO: 34 (SNP base bold):
TTGTCTGCTATGTTTGATGGTGTCTCCAAATCATTAGTATTGATG
CATGATTTGTCCGTCCGGGCTTTAATAACACGGTATCTGGCCATA
ACAGAAGTCT T GTCATTTCCAGCCGCCTCCTTGTTTGTGCCAG
AAACAGCATAATTCCGGATGAAACTATACAAGTTTTGACAACCCC
CTGCTTCAGGTGTCAATGAATTA

Chickpea plants, progeny thereof and/or part(s) thereof, are provided, which comprise a plurality of quantitative trait loci (QTLs) having a corresponding plurality of nucleic acid genetic markers that are associated with a plurality of phenotypic traits of seeds of the chickpea plant comprising at least one of a protein content trait, a fat content trait, a protein composition trait and a seed color trait, wherein the QTLs are genetic elements combined from different chickpea varieties by computationally supported breeding. Parts of the plants may include any of seeds, endosperm, ovules, pollen, cells, cell cultures, tissue cultures, plant organs, protoplasts, meristems, embryos, or combinations thereof.

In certain embodiments, QTLs 1 to 17 may comprise: QTL 1 with corresponding markers set forth in SEQ ID NOs 1 or 2, wherein QTL 1 is homozygous with respect to SEQ ID NO 1 or heterozygous, QTL 2 with corresponding markers set forth in SEQ ID NOs 3 or 4, wherein QTL 2 is homozygous with respect to SEQ ID NO 3 or heterozygous, QTL 3 with corresponding markers set forth in SEQ ID NOs 5 or 6, QTL 4 with corresponding markers set forth in SEQ ID NOs 7 or 8, wherein QTL 4 is homozygous with respect to SEQ ID NO 7 or heterozygous, QTL 5 with corresponding markers set forth in SEQ ID NOs 9 or 10, wherein QTL 5 is homozygous with respect to SEQ ID NO 9 or heterozygous, QTL 6 with corresponding markers set forth in SEQ ID NOs 11 or 12, wherein QTL 6 is homozygous with respect to SEQ ID NO 11 or heterozygous, QTL 7 with corresponding markers set forth in SEQ ID NOs 13 or 14, wherein QTL 7 is homozygous with respect to SEQ ID NO 13 or heterozygous, QTL 8 with corresponding markers set forth in SEQ ID NOs 15 or 16, wherein QTL 8 is homozygous with respect to SEQ ID NO 15 or heterozygous, QTL 9 with corresponding markers set forth in SEQ ID NOs 17 or 18, wherein QTL 9 is homozygous with respect to SEQ ID NO 17 or heterozygous, QTL 10 with corresponding markers set forth in SEQ ID NOs 19 or 20, wherein QTL 10 is homozygous with respect to SEQ ID NO 19 or heterozygous, QTL 11 with corresponding markers set forth in SEQ ID NOs 21 or 22, wherein QTL 11 is homozygous with respect to SEQ ID NO 24 or heterozygous, QTL 12 with corresponding markers set forth in SEQ ID NOs 23 or 24, wherein QTL 12 is homozygous with respect to SEQ ID NO 23 or heterozygous, QTL 13 with corresponding markers set forth in SEQ ID NOs 25 or 26, QTL 14 with corresponding markers set forth in SEQ ID NOs 27 or 28, wherein QTL 14 is homozygous with respect to SEQ ID NO 27 or heterozygous, QTL 15 with corresponding markers set forth in SEQ ID NOs 29 or 30, QTL 16 with corresponding markers set forth in SEQ ID NOs 31 or 32, wherein QTL 16 is homozygous with respect to SEQ ID NO 31 or heterozygous, and QTL 17 with corresponding markers set forth in SEQ ID NOs 33 or 34, wherein QTL 17 is homozygous with respect to SEQ ID NO 33 or heterozygous. In certain embodiments, the QTLs comprise at least three of QTLs 1 to 17.

The QTLs may be arranged in one or more cassettes comprising at least three of QTLs 1 to 17. In certain embodiments, referred to herein as cassette 1, the QTLs may comprise QTLs 1, 2, 3, 4 and 5, with QTL 3 being homozygous with respect to SEQ ID NO 5 or heterozygous. In certain embodiments, referred to herein as cassette 2, the QTLs may comprise QTLs 6, 7, 8, 9, 10, 11, 12, 13 and 14, with QTL 13 being homozygous with respect to SEQ ID NO 25 or heterozygous. In certain embodiments, referred to herein as cassette 3, the QTLs may comprise QTLs 7, 13 and 15, with QTL 13 being homozygous with respect to SEQ ID NO 26 or heterozygous and QTL 15 being homozygous with respect to SEQ ID NO 29 or heterozygous. In certain embodiments, referred to herein as cassette 4, the QTLs may comprise QTLs 2, 3, 13 and 17, with QTL 13 being homozygous with respect to SEQ ID NO 25 or heterozygous. In certain embodiments, referred to herein as cassette 5, the QTLs may comprise QTLs 3, 12, 13, 15 and 16, with QTL 3 being homozygous with respect to SEQ ID NO 6 or QTL 13 being homozygous with respect to SEQ ID NO 25 or heterozygous and QTL 15 being homozygous with respect to SEQ ID NO 30 or heterozygous.

In certain embodiments, the chickpea plant, progeny thereof or part thereof may have a protein content in the seeds of the plant which is at least 10%, 20%, 30% or 40% higher than of seeds of the CDC Orion cultivar. In certain embodiments, the chickpea plant, progeny thereof or part thereof may have a fat content in the seeds of the plant which is at least 10%, 15% or 20% lower than of seeds of the CDC Orion cultivar.

Table 3 illustrates the uniqueness of the disclosed chickpea varieties, as characterized by at least one of cassettes 1-5 and their combinations, according to some embodiments of the invention with respect to the world varieties. The number of disclosed chickpea varieties is indicated with respect to the combination of marker cassettes which characterize the respective chickpea varieties. The world varieties include all 3108 varieties, as disclosed below.

TABLE 3
Comparison of disclosed varieties included in the disclosed
cassettes with world (prior art) varieties.

| | | | | | Number of Varieties | |
|---|---|---|---|---|---|---|
| Cassette Combination | | | | | Disclosed | |
| 1 | 2 | 3 | 4 | 5 | (32 in total) | World |
| | | included | | | 12 | 0 |
| included | | | included | | 5 | 0 |
| | included | | | | 5 | 0 |
| | | | | included | 5 | 0 |
| included | included | | included | | 5 | 0 |

FIG. 5 is a high-level schematic illustration of a computationally supported breeding method 200, according to some embodiments of the invention. Method 200 may be at least partially implemented by at least one computer processor; computationally supported breeding method 200 is used to detect and combine QTLs from a plurality of chickpea varieties to develop the disclosed chickpea plants which are different than any of the parent varieties by virtue of the achieved phenotypical characteristics.

Computationally supported breeding method 200 comprises stages of trait discovery by growing and phenotyping a broad spectrum of varieties (stage 210), trait blending by developing hybridized lines through crossing the selected lines to mix and combine traits and selfing of the progeny in subsequent generations (stage 220), Target Product Genomic Code (TPGC) discovery by associating phenotypes and genotypes using derived linkage maps (stage 230), in silico selection to suggest candidate varieties (stage 240), breeding candidate varieties and selection of varieties based on the best TPGC potential (stage 250) and genomic code (GC) discovery to identify the most stable QTLs in hybridized progeny generation(s) (stage 260), as explained in detail below. TPGC discovery 230, in silico validation 240 and GC discovery 260 are based on computational algorithms that cannot be performed manually and provide the computational support for the judicious selection of the varieties that are generated and further crossed during the development process to yield disclosed chickpea plants. It is noted that during the discovery phase, the QTLs are derived in order to combine them (by hybridization) to create unique combinations of QTLs which do not exist in known world lines.

In certain embodiments, chickpea lines were bred to reach high protein, low fat and/or modified protein composition by collecting various chickpea lines worldwide, creating F2 linkage populations, applying intensive phenotyping and genotyping of thousands of chickpea lines, predicting of QTLs affecting the protein, protein composition and/or fat level traits, and establishing unique QTL marker combinations, termed “marker cassettes” herein, to characterize novel lines provided by the methods described herein and not existing in commercial or natural lines.

The breeding methodology was based on algorithms for deriving the Target Product Genomic Code (TPGC) to associate (i) the Target Product (TP) being defined in advance based on market requirements and including a set of desired attributes (traits) that are available in natural genetic variations; and (ii) the Genomic Code (GC) comprising set(s) of genomic regions that include quantitative trait loci (QTLs) that affect and are linked to the TP traits. The algorithms may be configured to calculate multiple genomic interactions and to maximize the genomic potential of specific chickpea plants for the development of new chickpea varieties. The breeding program was constructed to derive the TPGC, and then by crossing and selfing to achieve a chickpea product which contains the specific GC that corresponds to the required TPs.

Certain embodiments of the breeding process of developing lines, through crossing and successive generations of selfing comprise stages such as: (i) Trait Discovery, in which a broad spectrum of chickpea varieties from different geographies and worldwide sources are grown and phenotyped in order to identify new traits that can potentially be combined to create new chickpea varieties; (ii) Trait Blend, in which a crossing cycle is carried out based on phenotypic assumption(s), in which the different traits are mixed and combined. Initial trait cycle(s) are followed by additional cycle(s) to create F2 (and possibly higher generations) population(s) that provide the basis for algorithmic analysis for constructing the TPGC; (iii) TPGC Discovery, in which the chickpea plant(s) are phenotyped and genotyped to produce linkage map(s), discovering the relevant QTLs and deriving the TPGC; (iv) several line validation stages over several breeding seasons in which chickpea lines based on millions of in silico calculated variations (and/or selections) are grown and are used to define the initial varieties; (v) Trait TPGC Blend, in which accurate crossings are performed in order to calculate the most efficient way to reach the best TPGC. The crossings are performed after in silico selection from millions of combinations and are based, at least in part, on phenotype assumptions. And (vi) Consecutive algorithm-based GC discovery stage(s) applied to F2 (or higher generation) population(s) grown in additional cycle(s).

Defining the TP for high (or modified) protein and/or low fat chickpea varieties includes the development of high throughput methods for high (or modified) protein and/or low fat level identification.

In the following non-limiting example of the breeding process, Trait Discovery (i) was based on proprietary germplasm including hundreds of elite varieties and thousands of F2 individual plants and also 1500 different chickpea lines that were obtained from the Germplasm Resources Information Network (GRIN) and the Israel Gene Bank (IGB). These lines were used for the Trait Blend stage (ii), with crosses executed based on the potential for enrichment of genomic diversity to create new complex(es) of traits for the high (or modified) protein and/or low fat level as the initial step for the TP-directed breeding program for high (or modified) protein and/or low fat chickpea lines. The resulting F1 hybrids were later self-crossed to create F2 linkage populations that showed phenotypic segregation. The F2 populations were then planted in two different environments for discovering the TPGC (iii) that includes high (or modified) protein and/or low fat traits. After screening and deep phenotyping of 3318 individuals from 11 populations, a set of ca. 300 representatives were selected. The selected individuals from the F2 populations were further massively phenotyped for traits associated with high (or modified) protein and/or low fat, as detailed in the following description. The measurement results were summarized into the representative high (or modified) protein and/or low fat traits.

TPGC Discovery (iii) included genotyping ca. 2900 selected individual chickpea plants from 8 populations. The analysis was performed with a panel of 600 markers based on single nucleotide polymorphism (SNP) and directly designed based on the polymorphism found in the parental lines of the populations which were analyzed in depth using high throughput DNA sequencing technologies. The panel was designed to maximize the chance to have the largest number of common segregating SNPs in order to create highly similar linkage maps for all observed populations. The computation of linkage maps was executed on each linkage F2 population based on the genotyping results. Linkage maps were computed with MultiPoint, an interactive package for ordering multilocus genetic maps, and verification of maps was based on resampling techniques. Discovery of QTLs that are related to high (or modified) protein and/or low fat was carried out with the MultiQTL package, based on the linkage maps that were merged by

Multipoint and the F2 population phenotype data, and using multiple interval mapping (MIM). The significance and co-occurrences of the high protein level and protein content markers were evaluated using an algorithm that related the genotype-phase of each marker to respective QTLs and phenotypic traits in linkage maps of the eight F2 populations (also called “linkage F2 populations” herein) in each population, for populations in different environments. QTL significance was computed with permutation, bootstrap tools and FDR (false discovery rate) for total analysis. The linkage maps of all eight F2 populations and the information of the high (or modified) protein and/or low fat traits over all genotyped plants belonging to those populations were analyzed and used to predict the QTLs in a “one trait to one marker” model, in which for all markers that constructed the linkage maps, each trait was tested independently against each one of the markers. In the provided examples, altogether seventeen markers were found to be related to traits associated with high (or modified) protein, low fat and/or seed color components (sec Table 1 above). The occurrence of some high (or modified) protein and low fat markers in two or more linkage maps of the F2 population (repetitive markers) strengthened its significance as representative for QTLs for these traits.

In general, the eight linkage F2 populations presented different markers that related to high (or modified) protein, low fat and/or seed color. However, subsets of common markers were found to be shared by multiple populations and are referred to herein as marker cassettes.

It is emphasized that the breeding process is explained using non-limiting examples from a specific part of the breeding program, and is not limited to the specific populations and varieties derived by this specific part of the breeding program. For example, different F2 population may be bred and used to derive additional varieties that are characterized by one or more of the disclosed QTLs.

Following TPGC Discovery (iii), an in-silico breeding program (iv) was established to process the TPGC blend (including combinations of QTLs for different plants) to simulate and predict the genotypic states of self, cross-self and hybrid plant with respect to the QTLs and their predicted effects on each phase of the markers for the high (or modified) protein, low fat and/or seed color traits. The in-silico breeding program was constructed to yield millions of in silico selfing combinations which were bred and evaluated in-silico up to F4 to measure the potential for each of the genotyped plants to acquire the high (or modified) protein and/or low fat level in the right combination at the right phase. The analysis resulted in identifying ca. 393 F2 plants having the highest score for high (or modified) protein, low fat and/or seed color, which were then chosen for the actual selfing and cross-selfing procedures. The F3 seeds from these selected F2 plants were sown in plots in the subsequent growing season. Under this procedure, QTLs from different populations were combined to yield F3 plants containing new and unique cassettes of QTLs and resulting in high (or modified) protein, low fat and/or favorable seed color.

The high (or modified) protein and/or low fat chickpea lines were then validated as retaining the traits in the following generations by genotyping the F3 and some subsequent generation offspring to verify they maintained the identified marker cassettes. Specifically, the parental lines of linkage F2 populations together with 3108 different chickpea cultivars (landraces and old commercial varieties) were genotyped based on high (or modified) protein and/or low fat level markers of all populations. The cassettes detailed in Table 1 were found to wholly differentiate the developed high (or modified) protein and/or low fat lines from the rest of the chickpea cultivars screened.

The inventors note that none of the high (or modified) protein and/or low fat chickpea plants that were bred according to the methods described herein is naturally occurring; indeed, they were derived by highly complicated computationally-supported breeding methods 200 described above, in which the genotypes of multiple chickpea varieties were judiciously combined and analyzed, to discover and accumulate the recited QTL markers and corresponding phenotypic traits. Although the recited chickpea plants are not genetically modified by sequences originating from other species, they cannot be reached merely by natural processes, as is evident by the detailed and intentional breeding program that was applied to specifically measure required characteristics, detect corresponding markers using bioinformatics methods and combine the detected QTLs in the selected varieties by classic breeding approaches. The inventors note that due to the huge complexity of the breeding program, involving growing, selecting and breeding of hundreds of varieties over many generations in the field, and based on genetic analysis of the varieties and of the relations of QTL markers to phenotypic characteristics, this breeding process cannot happen merely by natural means and therefore cannot be considered a natural phenomenon. It is further noted that while the disclosed QTL markers are not heterologous to chickpea as a species, the identified QTLs are not present in the recited combinations in any of over 3108 landraces and old commercial varieties which were used as initial breeding stock, and that the QTL genomics of the chickpea plants has been significantly and judiciously modified by the breeding program.

Therefore, at the taxonomic level of the chickpea varieties, the high (or modified) protein and/or low fat chickpea plants may be considered hybridized in that the QTL markers are mixed and introduced from other chickpea varieties.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to the diagrams or to the corresponding descriptions. For example, flow need not move through each illustrated box or state, or in exactly the same order, as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some embodiments. Other possible variations, modifications, and applications are also within the scope of the invention. Accordingly, the scope of the invention should not be limited by what has thus far been described, but by the appended claims and their legal equivalents.

On ______, 2022, a deposit of at least ______ seeds of Equi-nom Ltd. chickpea line ______ was made by EQUI-nom Ltd. under the provisions of the Budapest Treaty with ______, and the deposit was given Accession No. ______. The deposit with be maintained in the international depository for a period of 30 years after the deposition. Should the seeds from the Equi-nom Ltd. chickpea line ______ deposited with international depository become unavailable, the deposit will be replaced by Equi-nom Ltd. upon request.

Chickpea plant, progeny thereof and/or part thereof EQUI-nom Ltd. chickpea line ______ is an exemplary variety which exhibits a protein content trait, a fat content trait, a protein composition trait and/or a seed color trait which make it commercially suitable.

1. A chickpea plant, progeny thereof or part thereof, comprising:
a plurality of quantitative trait loci (QTLs) having a corresponding plurality of nucleic acid genetic markers that are associated with a plurality of phenotypic traits of seeds of the chickpea plant comprising at least one of a protein content trait, a fat content trait, a protein composition trait and a seed color trait, wherein the QTLs are nucleic acid genetic elements combined from different chickpea varieties by computationally supported breeding, wherein the QTLs comprise at least one of QTLs 1 to 17 with corresponding nucleic acid markers set forth in SEQ ID NOs: 1 to 34.

2. The chickpea plant, progeny thereof and/or part(s) thereof according to claim 1, wherein QTLs 1 to 17 comprise:
QTL 1 with corresponding markers set forth in SEQ ID NOs 1 or 2, wherein QTL 1 is homozygous with respect to SEQ ID NO 1 or heterozygous,
QTL 2 with corresponding markers set forth in SEQ ID NOs 3 or 4, wherein QTL 2 is homozygous with respect to SEQ ID NO 3 or heterozygous,
QTL 3 with corresponding markers set forth in SEQ ID NOs 5 or 6,
QTL 4 with corresponding markers set forth in SEQ ID NOs 7 or 8, wherein QTL 4 is homozygous with respect to SEQ ID NO 7 or heterozygous,
QTL 5 with corresponding markers set forth in SEQ ID NOs 9 or 10, wherein QTL 5 is homozygous with respect to SEQ ID NO 9 or heterozygous,
QTL 6 with corresponding markers set forth in SEQ ID NOs 11 or 12, wherein QTL 6 is homozygous with respect to SEQ ID NO 11 or heterozygous,
QTL 7 with corresponding markers set forth in SEQ ID NOs 13 or 14, wherein QTL 7 is homozygous with respect to SEQ ID NO 13 or heterozygous,
QTL 8 with corresponding markers set forth in SEQ ID NOs 15 or 16, wherein QTL 8 is homozygous with respect to SEQ ID NO 15 or heterozygous,
QTL 9 with corresponding markers set forth in SEQ ID NOs 17 or 18, wherein QTL 9 is homozygous with respect to SEQ ID NO 17 or heterozygous,
QTL 10 with corresponding markers set forth in SEQ ID NOs 19 or 20, wherein QTL 10 is homozygous with respect to SEQ ID NO 19 or heterozygous,
QTL 11 with corresponding markers set forth in SEQ ID NOs 21 or 22, wherein QTL 11 is homozygous with respect to SEQ ID NO 24 or heterozygous,
QTL 12 with corresponding markers set forth in SEQ ID NOs 23 or 24, wherein QTL 12 is homozygous with respect to SEQ ID NO 23 or heterozygous,
QTL 13 with corresponding markers set forth in SEQ ID NOs 25 or 26,
QTL 14 with corresponding markers set forth in SEQ ID NOs 27 or 28, wherein QTL 14 is homozygous with respect to SEQ ID NO 27 or heterozygous,
QTL 15 with corresponding markers set forth in SEQ ID NOs 29 or 30,
QTL 16 with corresponding markers set forth in SEQ ID NOs 31 or 32, wherein QTL 16 is homozygous with respect to SEQ ID NO 31 or heterozygous, and
QTL 17 with corresponding markers set forth in SEQ ID NOs 33 or 34, wherein QTL 17 is homozygous with respect to SEQ ID NO 33 or heterozygous.

3. The chickpea plant, progeny thereof and/or part(s) thereof according to claim 1, wherein the QTLs comprise at least three of QTLs 1 to 17.

4. The chickpea plant, progeny thereof and/or part(s) thereof according to claim 3, wherein the QTLs are arranged in one or more cassette comprising the at least three of QTLs 1 to 17.

5. The chickpea plant, progeny thereof and/or part(s) thereof according to claim 4, wherein the one or more cassettes comprise at least cassette 1 comprising QTLs 1, 2, 3, 4 and 5, wherein QTL 3 is homozygous with respect to SEQ ID NO 5 or heterozygous.

6. The chickpea plant, progeny thereof and/or part(s) thereof according to claim 4, wherein the one or more cassettes comprise at least cassette 2 comprising QTLs 6, 7, 8, 9, 10, 11, 12, 13 and 14, wherein QTL 13 is homozygous with respect to SEQ ID NO 25 or heterozygous.

7. The chickpea plant, progeny thereof and/or part(s) thereof according to claim 4, wherein the one or more cassettes comprise at least cassette 3 comprising QTLs 7, 13 and 15, wherein QTL 15 is homozygous with respect to SEQ ID NO 26 or heterozygous.

8. The chickpea plant, progeny thereof and/or part(s) thereof according to claim 4, wherein the one or more cassettes comprise at least cassette 4 comprising QTLs 2, 3, 13 and 17, wherein QTL 13 is homozygous with respect to SEQ ID NO 25 or heterozygous.

9. The chickpea plant, progeny thereof and/or part(s) thereof according to claim 4, wherein the one or more cassettes comprise at least cassette 5 comprising QTLs 3, 12, 13, 15 and 16, wherein QTL 3 is homozygous with respect to SEQ ID NO 6 or heterozygous, QTL 13 is homozygous with respect to SEQ ID NO 25 or heterozygous and QTL 15 is homozygous with respect to SEQ ID NO 30 or heterozygous.

10. The chickpea plant, progeny thereof or part thereof according to claim 1, wherein a protein content of seeds of the plant is at least 40% higher than of seeds of the CDC Orion cultivar.

11. The chickpea plant, progeny thereof or part thereof according to claim 10, wherein a protein content of seeds of the plant is 40% higher than of seeds of the CDC Orion cultivar.

12. The chickpea plant, progeny thereof or part thereof according to claim 1, wherein a fat content of seeds of the plant is at least 10% lower than of seeds of the CDC Orion cultivar.

13. The chickpea plant, progeny thereof or part thereof according to claim 12, wherein a fat content of seeds of the plant is 20% lower than of seeds of the CDC Orion cultivar.

14. The chickpea plant, progeny thereof or part thereof according to claim 1, wherein the part thereof comprises any of a seed, an endosperm, an ovule, pollen, cell, cell culture, tissue culture, plant organ, protoplast, meristem, embryo, or a combination thereof.
